(19) United States
(12) Patent Application Publication
(10) Pub. No.: US 2012/0277593 A1
(43) Pub. Date: Nov. 1, 2012

(54) IMAGING BASED INTERFEROMETRIC PRESSURE SENSOR

(76) Inventors: Wuzhou Song, Ecublens (CH); Demetri Psaltis, Saint-Sulpice (CH)

(21) Appl. No.: 13/504,688

(22) PCT Filed: Oct. 28, 2010
(86) PCT No.: PCT/IB10/54890

§ 371 Date: Jul. 9, 2012

(30) Foreign Application Priority Data
Oct. 29, 2009 (IB) PCT/IB2009/054804

(51) Int. Cl. A61B 6/00

(52) U.S. Cl. 600/476

(57) ABSTRACT

An imaging based interferometric pressure sensor apparatus compromise a fluid pressure sensor unit (1) and an optical monitor (2). The disposable pressure sensor part comprises a rigid transparent cover plate 3 and a flexible diaphragm (4), in between forming an air-gap cavity (8). By illuminating the air-gap cavity (8) with a light source (6), the air-gap cavity generates an interference pattern which is captured by the optical imaging device (7). The pressure of the fluid (5) to be sensing is applied to flexible diaphragm, causing the deformation of the diaphragm and the variation of the air-gap thickness. Hence the interference pattern varies with pressure of fluid. The optical monitor (2) includes light source 6 and optical image device (7) which records the interference pattern from the fluid pressure sensor unit (1). The pressure of the fluid is measured by processing the captured image.

### FIELD OF INVENTION

The invention relates to pressure sensors for use in fluid pressure measurement.

### STATE OF THE ART

The market of single use medical devices is fast growing. There is an increasing demand of disposable pressure sensors which have broad applications in surgical procedure, patient monitoring and infusion controlling, etc.

For example, the disposable pressure sensor is used to directly measure the blood pressure of a human being or animal. The method is carried out by sticking a needle into an artery and connecting an external pressure sensor by means of liquid such as saline solution. Compared with the indirect measurement method, such direct measurement method enables the rapid, high precision measurement and long term continuous monitoring.

There is also an increasing trend in the industry and laboratory towards single use disposable technology. Pressure is an important process parameter that must be monitored in many bioprocess unit operations such as filtration, chromatography, and bioreactor production. For example, in flow filtration process, the monitoring and controlling the transmembrane pressure and the delta-pressure of the filter is necessary. An ideal solution for the end user would be to have a disposable pressure sensor that can be integrated with the disposable process assembly.

Electrical types of pressure sensors are known. For example in U.S. Pat. No. 5,105,820, the pressure is applied to a flexible diaphragm which contains a variable resistance element for sensing, the deformation of the diaphragm cause the resistance change which converts the voltage signal by a integrated electrical bridge circuit.

However, there exist several drawbacks of such electrical types of disposable pressure sensors:
1. There exists a potential failure of electrical contact. Stable electrical contacting between the disposable pressure sensor and the monitor is necessary during operation. The monitor here is for reading the signal from the disposable pressure sensor. For example, for the conventional types with electrical bridge circuit, four electrodes must simultaneous be connected to the monitor. No matter the cable with adapter or electrodes directly printing on circuit board, these conductive components within the disposable pressure sensor would increase its cost. In addition, there exist potential electrical contacting problem between the disposable pressure sensor and the monitor due to the contamination by the dust, oil, chemical, oxidation or abrasion As a result, the sensor may work improperly.
2. The sensor can be potentially affected an electromagnetic inference. Usually there is no electromagnetic shielding for the disposable pressure sensor. Error electrical signal can be induced by the external electromagnetic inference.
3. There exists additional cost due to the electrical circuit for temperature compensation on disposable pressure sensor.
4. There exists additional cost for package the electrical pressure sensor chip with the body of the disposable pressure sensor.

Several types of interferometric optical pressure sensors are known, as for instance disclosed in U.S. Pat. No. 6,738,145 where the optical fiber pressure sensors have a Fabry-Perot cavity at the end of an optical fiber. Variation in applied pressure alters the cavity length and hence the optical response of the etalon cavity. A monitor coupled to the optical fiber measures applied pressure by monitoring the optical characteristics of the etalon. Usually the monitor comprises a wide band light source coupled into the fiber and a spectrum analysis component for analyzing the reflection spectrum from the fiber. However, such optical fiber pressure sensors are seldom used as disposable pressure sensors, especially in the applications which can be done by the electrical disposable pressure sensors, due to the high cost of fabricating such fiber pressure sensor and the high cost of the monitor.

It would be desirable to have an even lower cost of disposable pressure sensors, and none contact is necessary hence avoiding the potential electrical contact failure.

### SUMMARY OF THE INVENTION

The present invention offers several advantages, in particular a very low cost optical interferometric disposable pressure sensor which is based on imaging method.

It relates to an imaging based interferometric fluid pressure sensor apparatus comprising a fluid pressure sensor unit 1 and a monitor 2 with an image sensor adapted to capture an interference pattern, said fluid pressure sensor unit 1 comprising a cavity 8 with an upper face consisting of a rigid transparent plate 3 and a bottom face consisting of a flexible diaphragm 4, said flexible diaphragm having its external side adapted to be in contact with a fluid 5.

The monitor preferably comprises a light source 6 and an image device 7.

In operation, the cavity is illuminated through the transparent plate 3 by the light source 6. An interference pattern is generated across the surface area of the air gap 8. The interference pattern is captured by the image sensor of the monitor 2. Variation of the fluid pressure alters the deformation of the flexible diaphragm 4 and hence the distribution of the air gap 8 thickness across the surface area which causes the variation of interference pattern. The interference pattern is captured by the image device 7 and further processed to calculate the pressure value.

Two or more such pressure sensor unit 1 can be connected in serious to form a differential pressure flow meter. Or several such air-gap cavities 8 can be integrated in single unit.

The pressure sensor unit can contain markers 9.

### BRIEF DESCRIPTION OF DRAWINGS

FIG. 1 illustrate the a preferred example of schematic of the whole apparatus (just as a summary picture)

FIG. 2 illustrates a preferred example of the configuration of the fluid pressure sensor unit 1, image device 7 and light source 6.

FIGS. 3, 4, 5 illustrate the examples of other different configuration of the image device 7 and light source 6.

FIG. 6 illustrates a preferred schematic cross-sectional structure of the a fluid pressure sensor unit 1.

FIG. 7 describes the interference mechanism of the air-gap cavity 8.

FIG. 8 is an example picture of the interference patterns by illuminating the air-gap cavity 8 with a narrow bandwidth light source.

FIG. 9 illustrates the examples of different cross-sectional structures on transparent plate 4.

FIG. 10 illustrates the examples of the different cross-sectional structures on the flexible diaphragm 4.

FIG. 11 illustrates an example that two air-gap cavities 8 are integrated into a single unit.

FIG. 12 illustrates the example of that the makers 9 locate inside and beside the air-gap cavity 8.

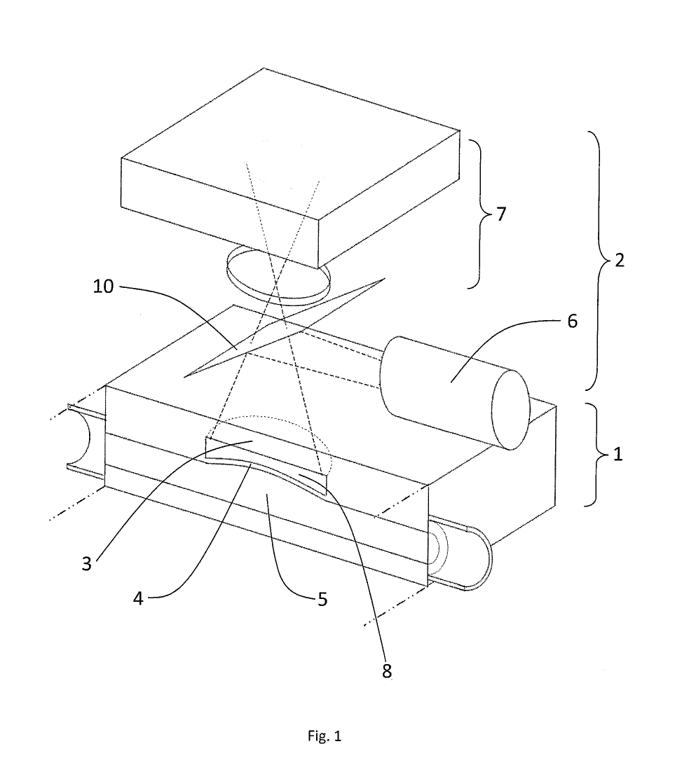

### DETAILED DESCRIPTION OF PREFERRED EMBODIMENTS

The invention will be better understood below with some examples.

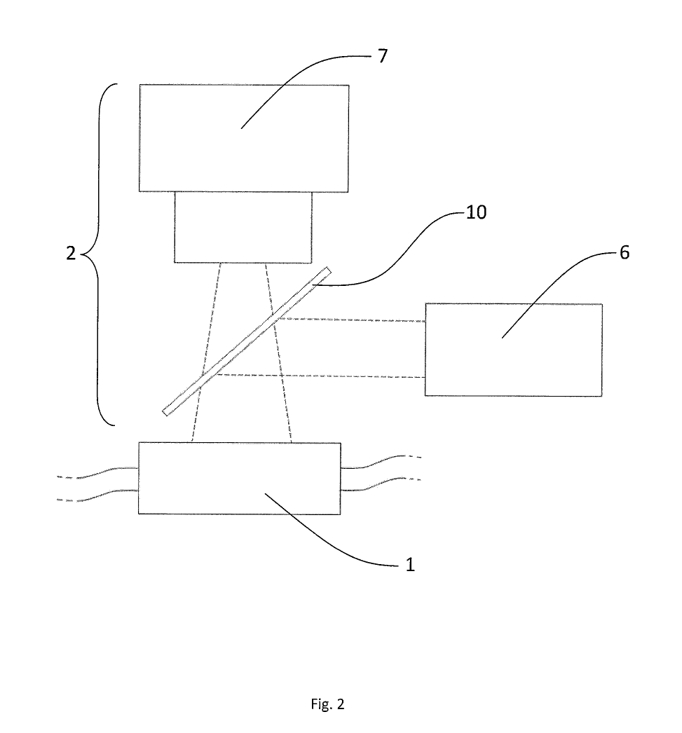

A preferred configuration of the whole system is illustrated in FIG. 2. It can be divided into two main parts: a (e.g. disposable) pressure sensor 1 and the monitor 2. Being preferably apermanent component, the monitor includes the light source 6 and image device 7.

As a preferred example, a narrow bandwidth light source can be used as light source 6. For example, the laser light source, LED light source or the light source containing narrow-linewidth bandpass filter can also be utilized as narrow bandwidth light source.

For example, the image device here includes the 1D or 2D image sensor and other passive optical components, such as lens or filter. As a preferred example, a CMOS (Complementary metal-oxide-semiconductor) image sensor can be also utilized as the image sensor. Furthermore, CMOS technology provides the ability to integrate DSP (digital signal processing) functions on the CMOS image sensor. Such tasks as noise filtering, pattern recognizing may be performed by the DSP. A narrow-linewidth bandpass filter can add into the image device to enable or enhance the visibility of the interference pattern.

In a preferred example of configuration, as illustrated in FIG. 2, a beam splitter 10 is used to reflect the illumination beam from light source 6 into the disposable pressure sensor part 1.

The light source 6 is not limited to single color. Several narrow bandwidth light sources with different wavelength centers can also combine together. For example, different color narrow bandwidth light source can turn on alternately for illumination.

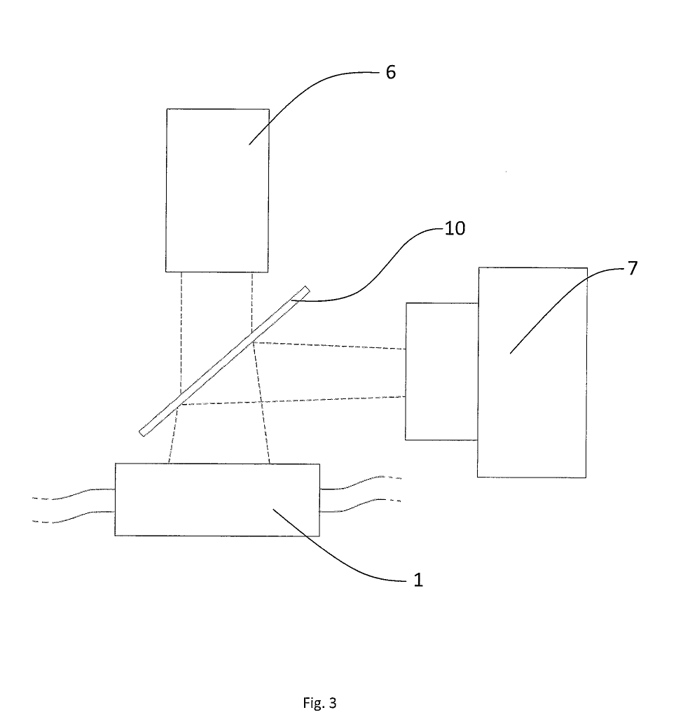

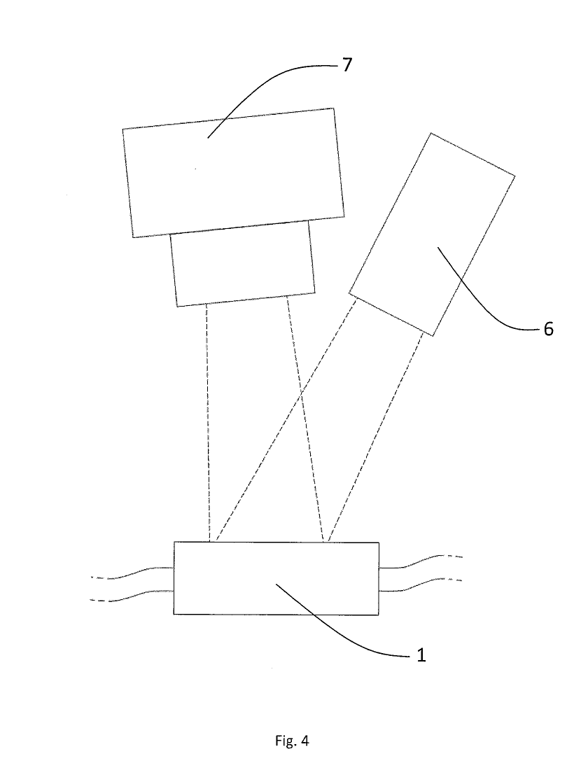

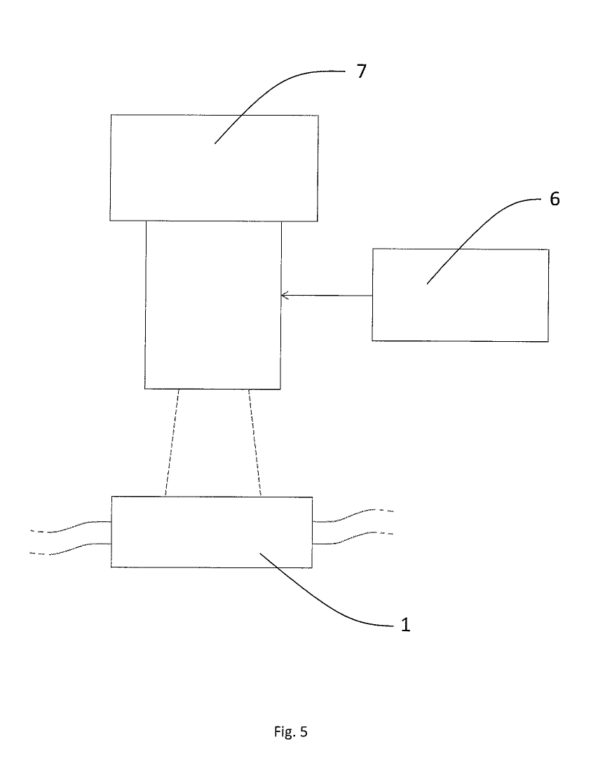

Other different configuration of the whole system is also allowed once the image device can capture the interference pattern from the fluid pressure unit 1. For example, in FIG. 3, the illumination beam directly goes through the beam splitter 10 and reaches the fluid pressure sensor unit 1. For example, in FIG. 4, the light source 6 and image device 7 are located closely without beam splitter. For example, in FIG. 5, the illumination beam couples into the image device 7, it also includes the configuration of that the light source 6 is directly integrated into the image device 7.

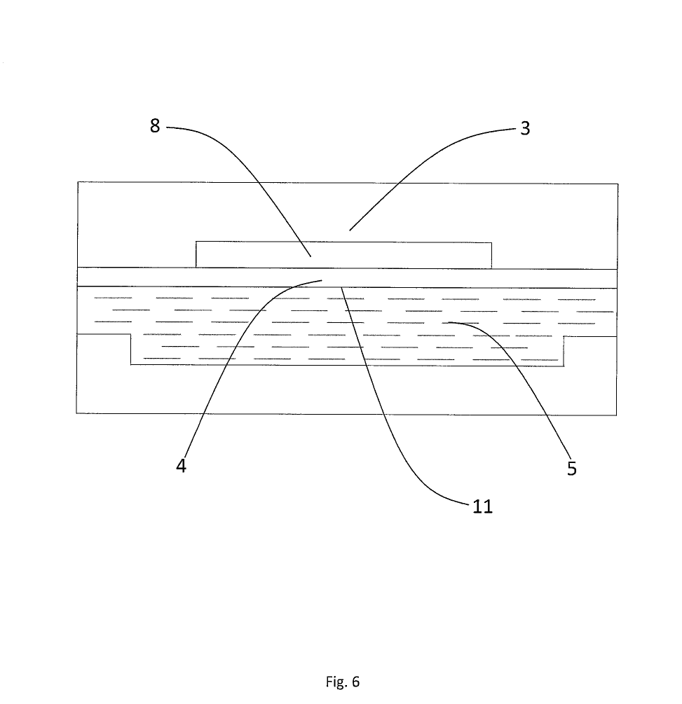

A preferred schematic cross-sectional structure of the fluid pressure sensor unit 1 is illustrated in FIG. 6. The key components of the fluid pressure sensor unit include a rigid transparent cover plate 3 and a flexible diaphragm 4. The transparent cover plate 3 allows the illumination light go through itself and reach the diaphragm 4. In between the transparent cover plate 3 and the flexible diaphragm 4, an air-gap cavity 8 is formed. On the back side 11 of the flexible diaphragm 4 (the back side is relative to the side of air-gap cavity) contacts with the fluid 5 of which the pressure is to be measured. As an exception, the fluid to be measured can also not necessarily directly contact the flexible diaphragm 4; in between there can exist some soft materiel to transfer the pressure force. For example, a soft material of polymer or gel can be coated on the back side 11 of the flexible diaphragm 4.

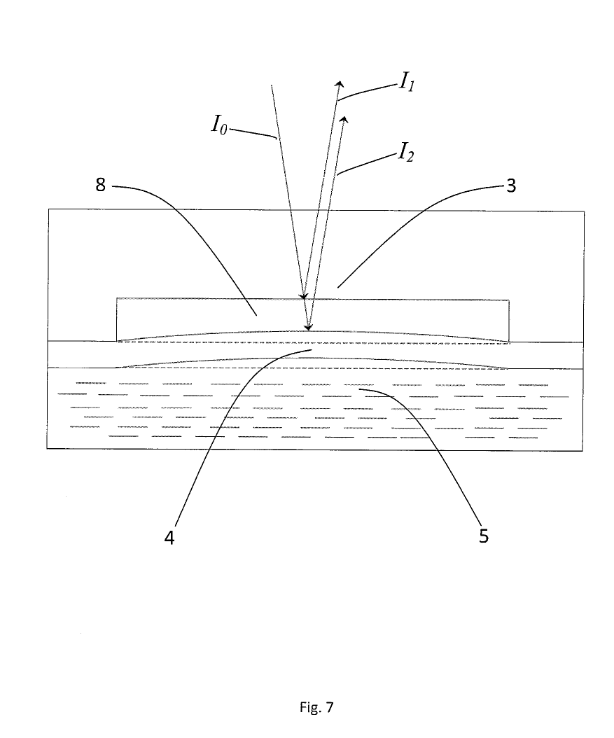

In operation, the pressure force from the fluid causes the deformation of the flexible diaphragm 4. The magnitude of the deformation of the diaphragm corresponds to pressure to be measured. As shown in FIG. 7 of the cross-sectional of an example schematic structure, the deformation of the flexible diaphragm changes the air-gap thickness and causes the variation of the thickness distribution of air-gap cavity 8. When the air-gap cavity is illuminated by incident beam I₀, the reflected light I₁, I₂ from two surfaces (I₁ from the cover plate-air interface, I₂ from the air-flexible diaphragm interface) interfere constructively or destructively, depending on the air-gap thickness. As the air-gap thickness is not uniform, an interference fringe pattern appears on the air-gap cavity 8. From the interference pattern, the relative air-gap thick distribution across the cavity can be estimated; hence the pressure can be measured.

The inside of air-gap 8 can be also filled with gas, liquid, in vacuum state or connecting to the atmosphere. As a preferred example, there is vent hole connecting the air-gap cavity to the atmosphere.

In the top view, the air-gap cavity 8 can be any shape.

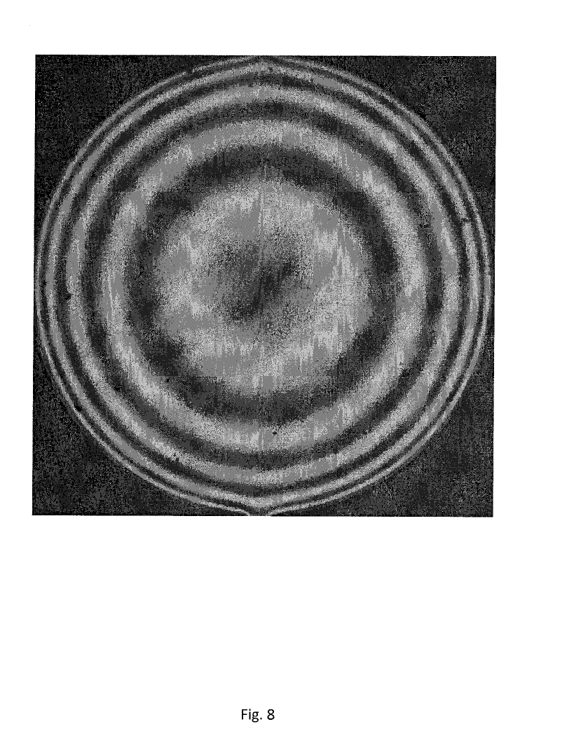

An example picture of the interference pattern is shown in FIG. 8. In this case, the air-gap cavity 8 is according the design in FIG. 6 and has circular shape in top view.

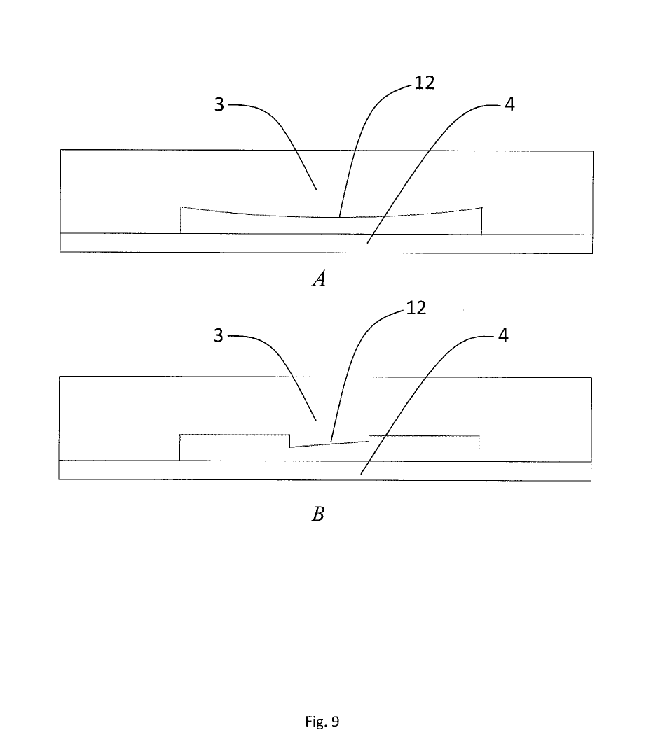

The inner surface 12 (towards the air-gap) of the cover plate 3 can have different cross-sectional structure. Different shape of the inner surface generates different patterns. For example, it has flat smooth surface as illustrated in FIG. 6. For example, it has convexity shape as illustrated in FIG. 9A. For example, it has step structure in FIG. 9B.

For example, the cover plate 3 can be made of polymer material fabricating with molding process.

Without coating on the inner surface 12 of the transparent cover plate, the light can reflect at the inner surface due to the refractive index contrast between cover plate and air (or the liquid filled inside). The inner surface 12 of the cover plate 3 can also be coated with reflective material to enhance the reflectivity. For example, single or multilayer of metal or dielectric material can deposit on the inner surface of the cover plate.

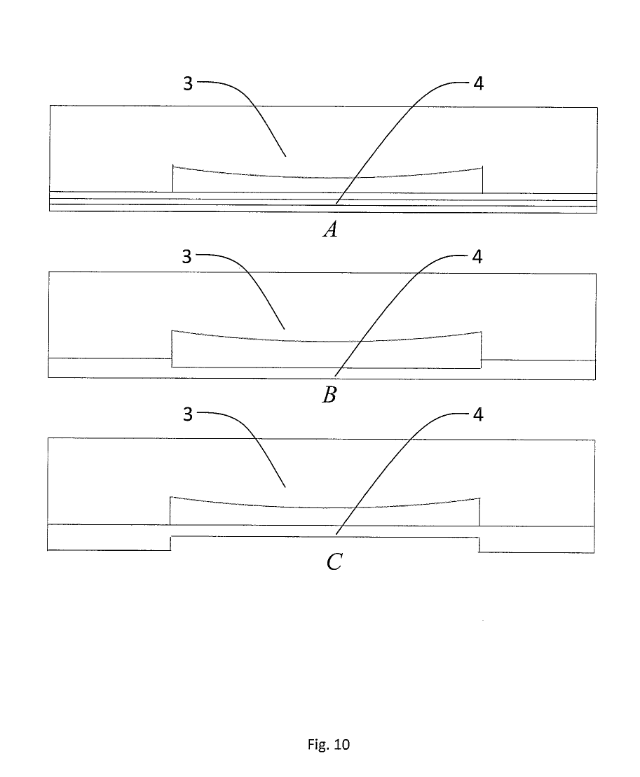

The flexible diaphragm 4 is made of single or composite material. For example, the FIG. 10A illustrate an example of that the diaphragm has multiple layer structure. For example, one layer can be a thin metal or dielectric film to enhance the reflectivity. For example, one layer can be a light absorption layer for eliminating the light interfering from the fluid chamber. For example, one layer can be made of a materiel with good elastic property.

The flexible diaphragm 4 can has different cross-sectional shape. For example, as illustrated in FIGS. 10B and C, there is a concave structure on each side of the diaphragm.

For example, the flexible diaphragm 4 has one layer structure which is made or one of such material that has good and stable elastic property: plastic, elastomer, glass, quartz or silicon.

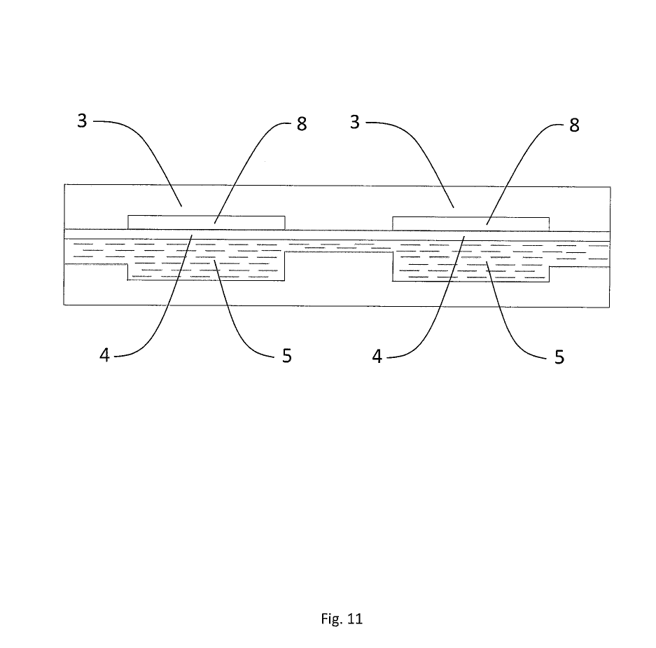

Two of more air-gap cavities can be integrated in a single device. For example, as illustrated in FIG. 11, two air-gap cavities 8 are connected in series inside one unit which can be functioned as a differential pressure flow meter. The flow rate can be calculated by measuring the pressure drop across the channel connecting the two air-gap cavities.

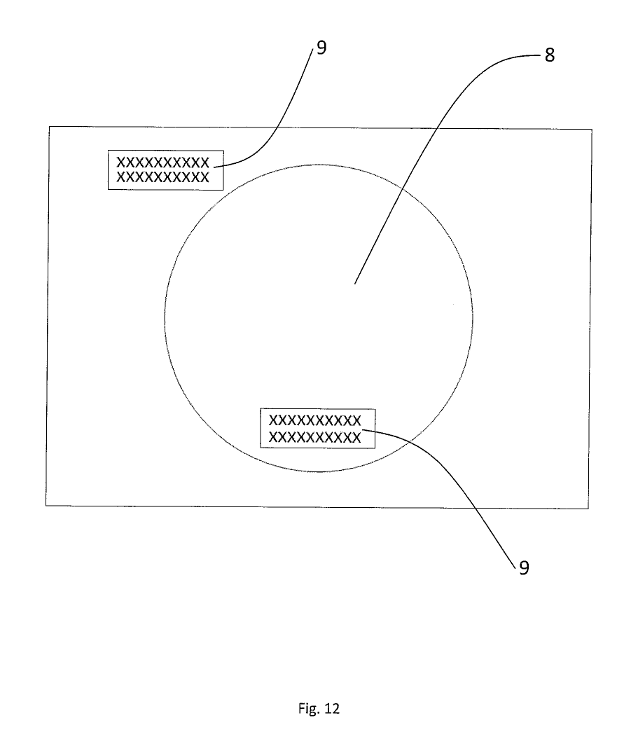

Markers 9 can be added inside the pressure sensor unit 1. These markers 9 can be captured by the image device of the monitor 2 and transfer the information. For example, as schematic illustrated in FIG. 12. For example, the markers can be text, numbers, dots, barcode or other pattern; they represent the information of the identity of the device, production information and calibration information. For example, these markers can be written by laser marker scribing machine during the production process.

By illuminating the air-gap cavity 8 with a narrow bandwidth light source, the air gap generates an interference pattern which is captured by the optical imaging device 7. From the captured image of the pattern, the pressure of the fluid can be measured.

The narrow light source includes laser diode, LED or narrow-linewidth bandpass filter plus wide band light source.

Several narrow bandwidth light sources with different wavelength centers may be combined together as the light source 6 inside the monitor 2.

The image device 7 comprises the 1D or 2D image sensor and other passive optical components, such as lens or filter.

A CMOS image sensor may be used for recording the image of interference pattern and markers 9.

A narrow-linewidth bandpass filter can add into the image device 7 to enable or enhance the visibility of the interference pattern.

Both side of the air-gap cavity 8 may be coated with metal or dielectric materials.

A soft material can be coated on between the flexible diaphragm 4 and fluid to be measured.

Markers 9 may be added inside the pressure sensor unit 1 which can be captured by the image device 7 in the monitor 2.

The air-gap cavities 8 may also be filled with gas, liquid, in vacuum state or connected to the atmosphere. In a preferred example, the air-gap cavity connects to the atmosphere through a vent hole.

The flexible diaphragm 4 may be made of single or composite material.

The flexible diaphragm 4 may have one of layer structure which is made or one of such material that has good and stable elastic property: plastic, elastomer, glass, quartz, ceramic or semiconductor material.

The inner surface 11 (towards the air-gap) of the cover plate can have different structure.

The cover plate 3 may be made of polymer (including plastic) material fabricating with molding process.

Two or more air-gap cavities 8 may be integrated in a single unit for measuring the flow rate.

The markers 9 may be text, number, dots, barcode or other pattern; they represent the information of the identity of the device, production information, calibration information or others.

The markers 9 may be imprinted or written by laser scribing machine during the production process.

The entire pressure sensor apparatus is primarily designed for the disposable application and liquid pressure sensing. However, it is not restricted to the disposable application or liquid pressure sensing. It is also applicable for the permanent application and gas pressure sensing.

The pressure sensor according to the invention can also be used for fluidic viscosity measurement. In that case the flow rate has to be known. Adavantageously the pressure sensor can be integrated into a chip.

Finally, it should be mentioned that the present invention is not limited to the examples discussed previously.

1. An imaging based interferometric fluid pressure sensor apparatus comprising a fluid pressure sensor unit 1 and a monitor 2 with an image sensor adapted to capture an interference pattern, said fluid pressure sensor unit comprising a cavity 8 with an upper face consisting of a rigid transparent plate 3 and a bottom face consisting of a flexible diaphragm 4, said flexible diaphragm 4 having its external side adapted to be in contact with a fluid 5.

2. A fluid pressure sensor apparatus according to claim 1 wherein said cavity 8 is filled with air.

3. A fluid pressure sensor apparatus according to claim 1 wherein said fluid pressure sensor unit 1 is disposable.

4. A fluid pressure sensor apparatus according to claim 3 wherein said monitor 2 is not single-use.

5. A fluid pressure sensor apparatus according to claim 1 wherein said pressure sensor unit 1 contains markers 9.
